(19) United States
(12) Patent Application Publication
(10) Pub. No.: US 2009/0035800 A1
(43) Pub. Date: Feb. 5, 2009

(54) Novel Use of Fluorescence Resonance Energy Transfer

(76) Inventors: T.J. Aartsma, Leiden (NL); G.W. Canters, Leiden (NL)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/722,762

(22) PCT Filed: Dec. 23, 2005
(86) PCT No.: PCT/EP05/14215

§ 371 Date: Jun. 25, 2007

(30) Foreign Application Priority Data
Dec. 24, 2004 (EP) 04258120.7

(51) Int. Cl. C12Q 1/32; G01N 21/64; C12N 9/00; C12M 1/00; C12N 9/02; C12Q 1/26

(52) U.S. Cl. 435/26; 436/172; 435/25; 435/183; 435/189; 435/288.7

(57) ABSTRACT

A novel use of Fluorescence Resonance Energy Transfer wherein a labelled protein comprising a Fluorescent energy donor label and at least one energy acceptor moiety capable of accepting energy from the donor label by Förster energy transfer is exposed to incident electromagnetic energy to excite the donor moiety and the fluorescence emission of the donor is measured. The or each energy acceptor moiety has a more and less active energy acceptor state and the level of quenching of donor fluorescence is indicative of this state. The energy acceptor moiety may be converted between its states by a redox reaction, optionally involving a partner redox protein. A novel system comprising the labelled protein, a redox partner protein, ‘a light source for imposing incident light at the excitation wavelength for the fluorescent label and a light detector capable of detecting the fluorescence emitted by the label may be used in biosensors and/or to monitor enzymatic turnover.

The present invention relates to a novel use of Fluorescent Resonance Energy Transfer (FRET) to monitor the activity of a donor-acceptor pair on a protein.

### BACKGROUND OF THE INVENTION

Fluorescence detection is a popular method for visualising and monitoring the activity and function of biomacromolecules because of its unmatched sensitivity. Often, dual wavelength fluorescence detection of a donor-acceptor pair is used, where fluorescence energy transfer (FRET) allows registration of conformational dynamics that is very sensitive to donor-acceptor distance and relative orientation [1].

FRET is based on a distance-dependent interaction between the electronic excited states of two dye molecules in which excitation is transferred from a donor molecule to an acceptor molecule without the emission of a photon. This process is known as Förster energy transfer. The efficiency of FRET is dependent on the inverse sixth power of intermolecular separation [2], making it useful over distances comparable with the dimensions of biological macromolecules. When FRET is used as a contrast mechanism, colocalisation of proteins and other molecules can be imaged with spatial resolution beyond the limits of conventional optical microscopy [3].

In order for FRET to occur the donor and acceptor molecules must be in close proximity (typically 10-100 Å), the absorption spectrum of the acceptor must overlap with the fluorescence emission spectrum of the donor, and the donor and acceptor transition dipole vectors must be approximately parallel, or at least not orthogonal.

When the donor and acceptor dyes are different, FRET can be detected by the appearance of sensitized fluorescence of the acceptor or by quenching of donor fluorescence. Non-fluorescent acceptors such as dabcyl have the particular advantage of eliminating the potential problem of background fluorescence resulting from direct (ie. non-sensitized) acceptor excitation.

Probes incorporating fluorescent donor-non-fluorescent acceptor combinations have been developed. Matayashi et al [4] detect proteolysis of a HIV protease substrate by elimination of the FRET signal between a EDANS fluorophore and a dabcyl quencher. Tyagi et al [5] describe probes that fluoresce when nucleic acid hydridisation causes the fluorophore and quencher to be separated. These probes are all based on the distance-dependence of quenching. A reagent consisting of a fluorophore and a quencher optionally connected to each other through a linker has been disclosed [6]. This conjugate reagent does not comprise a labelled protein.

### SUMMARY OF THE INVENTION

The present invention uses FRET in a novel way, wherein the change in quenching is not due to a change in donor-acceptor distance or relative orientation.

There is provided a method of fluorescence detection of a donor-acceptor pair in which a labelled protein comprising a fluorescent energy donor label and at least one energy acceptor moiety capable of accepting the energy from the donor label by Förster energy transfer, thereby quenching the donor fluorescence, is exposed to incident electromagnetic energy to excite the donor moiety and the fluorescence emission of the donor is measured, characterised in that the or each energy acceptor moiety has a more active and less active energy acceptor state and in that the level of quenching of fluorescence is indicative of the state of the or each energy acceptor moiety. The switch between the more and the less active states of the energy acceptor moiety may be the result of a chemical or biochemical reaction involving the energy acceptor moiety.

There is also provided in the invention a labelled protein comprising a fluorescent energy donor label and at least one energy acceptor moiety capable of accepting energy from the donor label by Förster energy transfer characterised in that the or each energy acceptor moiety is preferably non-fluorescent and has a more active and a less active energy acceptor state between which the moiety may be reversibly converted.

There is provided in the invention a system comprising the protein discussed above and a redox partner protein, a light source for imposing incident light at the excitation wavelength for the fluorescent label and a light detector capable of detecting the fluorescence emitted by the label.

The system may be used in a biosensor with dramatically improved sensitivity compared to current biosensors which are based on the sensing of an electric current by using electronically coupled redox enzymes and electrodes.

Sensitivity is a critical factor for biosensor applications since it determines the minimum concentration at which the analyte can be detected. Typical electrochemical biosensors, based on amperometric read-out, have a detection level in the order of 10⁻⁶M. The use of FRET according to the present invention lowers the detection level of redox activity to the sub-nanomol/L range, which allows the observation of single molecules under suitable conditions.

### DETAILED DESCRIPTION OF THE INVENTION

The present invention provides a labelled protein containing at least one energy acceptor moiety which has a more and a less active energy acceptor state. The activity of the or each energy acceptor moiety is related to its ability to accept the energy from the donor label and quench the donor's fluorescent emission. It therefore follows that the more active state accepts energy more readily than the less active state and consequently quenches more of the donor's fluorescence. In a preferred embodiment of the invention the less active energy acceptor state is completely inactive and will therefore quench no donor fluorescence. This facilitates experimental detection of the state of the energy acceptor moiety.

The or each energy acceptor moiety of the labelled protein according to the present invention may be reversibly converted from its more active state to its less active energy state and vice versa. This may occur by a chemical/biochemical reaction or a change in the environmental conditions surrounding the acceptor molecule. For example, an enzymatic reaction may occur which alters the energy-absorbing ability of the acceptor molecule. Suitable enzymes include proteases, kinases, phosphatases, glycosylases, oxido-reductases and transferases. Alternatively, a pH change in the external medium may switch the energy acceptor from its more to its less active form.

The or each energy acceptor may also be non-reversibly converted between its more and less active states. This would be of use in an assay where a one-off experiment is sufficient.

The fluorescent energy donor label of the protein of the present invention may be a fluorescent dye on the protein surface. This dye may be covalently attached to a specific protein residue or be an intrinsic property of the protein molecule.

Suitable fluorophores for labelling the proteins are common in the art, and include Cy5, Cy3 (Trademark name of dyes from Amersham Biosciences), Alexa Fluor (488, 568, 594 and 647), Tetramethylrhodamine (TMR) and Texas Red, (all obtainable from Molecular Probes, Inc). These may be functionalised either with a maleimide linker for binding to a free thiol group on the protein, or with a succinimydyl ester for binding to a free protein amine group. FIG. 1 shows how a dye may be covalently linked to a thiol. In this case the reaction involves oxidative coupling of a cysteine thiol group with a maleimide derivative of Cy5.

A typical method of labelling the protein of the present invention would include the steps of 1) adding bicarbonate to a solution of the protein of the present invention, 2) adding ˜100 μl of protein to the functionalised dye, 3) incubating for one hour, 4) stopping the reaction, 5) incubating for a further 15 minutes and 6) purifying the conjugate on a suitable column using, for example, 0.5M NaCl in water as an eluent. The purifying step ensures that most of the proteins become labelled with a dye molecule, thereby increasing the sensitivity of the method. The concentration of protein used according to the present invention should be high enough to allow detection of fluorescence, preferably 0.01 to 10 μM, more preferably 1 to 2 μM.

Alternatively, the protein used in the invention may be intrinsically fluorescent, such as the Aequora-related green fluorescent protein. Fluorescent proteins whose amino acid sequences are either naturally occurring or engineered by methods known in the art are included within the scope of the invention. Fluorescent proteins can be made by expressing nucleic acids that encode fluorescent proteins, such as wild-type or mutant Aequorea green fluorescent protein, in an appropriate cellular host [7].

It is an essential requirement of FRET that the absorption spectrum of the or each acceptor moiety overlaps with the fluorescence emission spectrum of the donor moiety. In the process according to the present invention, incident light is supplied by an external source, such as an incandescent lamp or a laser and should be of appropriate wavelength to be absorbed by the dye moiety, creating an excited electronic singlet state (S₁). Fluorescence is then emitted as the fluorophore returns to its ground state (S₀). The invention requires that this fluorescence is quenched by the acceptor moiety, and for this to occur the acceptor must absorb in the spectral region at which fluorescence is occurring.

Spectral overlap can be defined quantitatively using the expression for the spectral overlap integral:

J(λ)=∫E_{A}(λ)·F_{D}(λ)·λ⁴·dλcm³M⁻¹ Equation 1

where E_{A} is the extinction coefficient of the acceptor and F_{D} is the fluorescence emission intensity as a fraction of the total integrated intensity.

It is important to compare the emission spectrum of the dye with the absorption spectrum of the or each acceptor moiety when selecting a dye and acceptor combination for use in the method of the present invention. FIG. 2 shows the spectral overlap integral for the emission spectrum of dye Cy5 (grey line) with the absorption spectrum of oxidised azurin (dashed.) The shaded area indicates the region of overlap.

The acceptor moiety is preferably non-fluorescent. However, acceptors which fluoresce at wavelengths different to the donor fluorescence wavelength may also be used, as may acceptors which fluoresce at the same wavelength as long as they do so with a different quantum efficiency.

FRET is a strongly distance-dependent process. The energy transfer efficiency, E, as a function of distance R between the donor and the acceptor is given by equation 2 [8]:

$$E = \frac{{F}_{o} - {F}_{r}}{{F}_{o}} = \frac{{R}_{o}^{6}}{{R}_{o}^{6} + {R}^{6}} \qquad Equation 2$$

Fᵣ and Fₒ denote the fluorescence intensity in the presence and the absence of the quencher, respectively. Rₒ is a characteristic distance that depends on the refractive index, n, the spectral overlap between donor and acceptor bands, J(λ), the fluorescence quantum yield of the donor, Q_{D}, and the relative orientation of the optical transition moments of donor and acceptor as reflected by an orientation factor κ². This equation is used in example 3 to calculate the quenching rate for azurin, a protein which demonstrates many aspects of the present invention.

The labelled protein of the present invention may be an enzyme. In a preferred embodiment of the invention the enzyme is a redox enzyme and conversion from the more to the less active state (and vice versa) occurs via a redox reaction. In this preferred embodiment a redox co-factor with variable oxidation states may function as the energy acceptor. Many proteins found in nature are metalloproteins containing an intrinsic redox cofactor, like a flavin, a PQQ group or a transition metal, which will function as the energy acceptor moiety of the present invention. Electron transfer reactions belong to the most fundamental processes of life and for such reactions metalloproteins are highly suitable catalysts because of the ability of transition metals to exist in more than one stable oxidation state. Examples of metal ions commonly found in nature with variable oxidation states include copper and iron.

The method proposed by this embodiment of the present invention takes advantage of the fact that the optical characteristics of the redox co-factor vary with a change of its redox state. Fluorescence resonance energy transfer (FRET), then is a mechanism whereby a change in redox state of the co-factor translates into a change in fluorescence intensity of the label. Sensitivity has been shown to be sufficient to observe and monitor individual redox proteins. The method may eventually find use in sensitive fluorescent detection of electron transfer events and of enzymatic turn-over and also in biosensors, high-throughput screening and nanotech-based electronics.

The metalloprotein discussed above may belong to the family of blue copper proteins, or be a conjugate of one or more of these proteins, giving a fusion protein.

Members of this family include copper-containing laccases and oxidases and the small blue copper proteins, for example azurin, from Pseudomonas aeruginos, pseudoazurin from Alcaligenes faecalis, plastocyanin from Fern Dryopteris crassirhizoma and amicyanin from Paracoccus versutus. Haem containing proteins like cytochrome c550 from P. versutus and flavin-containing proteins like flavadoxin II from A. vinelandii may also be used in the present invention. Furthermore, the method may be used with redox enzymes, for example, methylamine dehydrogenase (MADH) from Paracoccus denitrificans, Nitrite reductase (NiR) from Alcaligenes faecalis, tyrosinase and Small Laccase (SLAC) from streptomyces coelicolor. Of these examples azurin will be used to exemplify the embodiments of the invention.

Azurin is a 14 kDa extensively studied protein carrying a single copper ion at its redox active centre. In its oxidised (Cu²⁺) form the protein displays a strong (ε, absorption coefficient=5.6 mM⁻¹ cm⁻¹) absorption in the 550-650 nm range (see FIG. 2), which corresponds to a π-π* transition of the Cu site, involving mainly the d_{x2-y2} orbital on the Cu and a 3 p orbital on the Cys112 sulfur. This absorption disappears when the Cu site is reduced because in the reduced (Cu⁺) form the Cu has a d¹⁰ electronic configuration and the optical absorption spectrum lacks conspicuous features (ε<10M⁻¹, cm⁻¹).

This pronounced change of absorption spectrum will strongly modulate the fluorescence properties of a FRET donor-acceptor pair, with the Cu-site as the energy acceptor and a dye-label, suitably linked to the protein, as the fluorescence donor. When Cu is in the oxidised state the fluorescence of the dye is strongly quenched as a result of the energy transfer to the π-π* excited state of the Cu site (which is non-fluorescing itself), whereas with the Cu in the reduced state the fluorescence is essentially uninhibited since the π-π* transition is absent. Thus the fluorescent dye acts as a passive “beacon” which is off (i.e. quenched) in the oxidised and on (not quenched) when Cu is in the reduced state in the protein.

The method of the present invention can also involve physiological partner proteins. In this embodiment the labelled protein docks with, for instance, a redox partner protein to/from which it donates or accepts electrons. The partner protein converts the energy acceptor moiety between its two states. The redox partner protein may be an enzyme capable of oxidising or reducing substrates where upon the labelled protein is switched between its states. The level of quenching in this case is indicative of the extent of the enzymic redox reaction and may be used to detect the presence or level of substrate. Table 1 lists a selection of systems which can be studied using the method of the present invention involving redox partner proteins. This aspect of the invention is detailed further in example 3.

TABLE 1

| Enzyme | Protein Partner | Detects |
|---|---|---|
| Nitrite reductase | (Pseudo)azurin, | Nitrite (NO₂⁻/NO) |
| | (p)Az | |
| Cytochrome p450 | Flavodoxin (FLD) | Aromatic compounds |
| Methylamine dehydrogenase | Amicyanin | Methylamine |
| Cytochrome c550 | Amicyanin | Various |

The partners of amicyanin are methylamine dehydrogenase (MADH) and cytochrome c550. The cyt-c550 functions as an electron shuttle and passes the electrons it receives from amicyanin on to other members of the electron transfer chain, i.e., respiratory enzymes like the membrane bound aa₃ cytochrome oxidase. The function of cyt c550 resembles that of amicyanin in that it accepts and passes on electrons.

Mutants of the wild-type proteins included within the scope of the present invention may also be prepared. These are useful to extend the range of substrates which may be detected. The mutants may be engineered using a directed evolution approach based on random PCR and a new screening procedure based on the fluorescence detection of NADPH consumption by P450 BM3 in whole E. coli cells (patent application pending.) As an example, nitrite reductase (NiR) and pseudoazurin (pAz) are considered in more detail. The copper enzyme nitrite reductase (NiR), eg from the bacterial source Alcaligenes faecalis, is part of the denitrification cycle, and reduces NO₂⁻ (nitrite) to NO (nitric oxide). The cupredoxin pseudoazurin (pAz; from the same bacterial source) functions as the electron donor in vivo to NiR. The electron transfer process is schematically represented below. The scheme shows an embodiment in which pAz is bound to e.g. a peptide modified gold electrode [9] or an indium doped tin oxide (ITO) electrode.

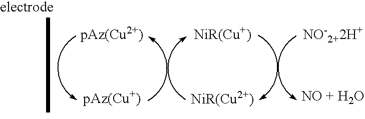

Either pAz or NiR can be labelled with a suitable fluorophore at a position on the protein surface. Upon excitation of the label fluorescence quenching would take place when the type 1 Cu site is in the oxidised (Cu(II)) state, but would not take place when the Cu is reduced. The change in the fluorescence signal may be used to monitor the transfer of electrons between the partner proteins. No change is to be expected in the absence of substrate.(NO₂⁻ in this case.)

Since Förster transfer depends on an overlap of the fluorescence spectrum of the donor with the acceptor, it can be calculated (see example 2) that the Förster radius (the distance at which FRET is 50% efficient—i.e. half of the donors are deactivated) of the oxidised type 1 Cu site for a typical fluorescent label is 30-40 Å. For efficient quenching upon reduction of the Cu site, the fluorescent label should be within this distance of the Cu site. PAz can thus be labelled anywhere on the protein surface since the size of this protein (diameter of approximately 25 Å) is less than the Förster radius. The shortest distance that can be achieved, without affecting the partner's docking site of either pAz or NiR, is about 15 Å. At this distance, fluorescence quenching by the oxidised type 1 Cu is virtually 100%, providing zero-background detection of the reduced state.

The Förster distance can be tuned to achieve energy transfer to only one of the two type 1 Cu sites in the pAz/NiR docked assembly by appropriate choice of the location of the label on the protein surface, so that one site is well within the Förster radius and the other is not (the two type 1 Cu sites in the docked complex are 15-18 Å apart).

The method is not only applicable to proteins that contain a redox-active type 1 Cu-site, but also to other proteins with co-factors that exhibit comparable changes in the absorption spectrum upon a change of redox state or another biochemical variable.

Partner proteins may be labelled with dyes that fluoresce at different wavelengths and that are quenched by different redox acceptor moieties, so that the dynamics between the two redox sites in the docked protein complex may be monitored by dual wavelength detection. Suitable fluorophores for labelling the proteins are common in the art, and have been previously listed in the application.

The present invention also includes a system comprising a protein according to the present invention, optionally together with a partner protein, a light source for imposing incident light at the excitation wavelength for the fluorescent label and a light detector capable of detecting the fluorescence emitted by the label. The system may additionally require wavelength filters for isolating emission photons from excitation photons. The detector of this system registers emission photons and produces a recordable output, which is preferably an electrical signal or a photographic image. Fluorescence instruments which may be used in the system of the present invention include spectrofluorometers, fluorescence microscopes, fluorescence scanners and flow cytometers.

In a preferred embodiment of the invention the protein is bound to a transparent substrate and total internal reflection is used to excite the surface-bound molecules to obtain a high signal-to-background ratio, and to achieve selectivity of excitation of surface bound particles. The transparent electrodes may be formed from materials common in the art, such as an SnO₂ coated glass substrate. For surface immobilisation of the protein engineered cysteines or His-tags may be used. When the system comprises a partner protein in addition to the first protein, preferably one of the proteins is bound to the transparent substrate. The other protein member may be freely diffusing in the medium surrounding the substrate.

When the or each energy acceptor moiety is redox-active, surface assembly of the redox proteins onto electrodes is preferred in order to achieve optimal electrochemical performance by direct electron transfer to and from the electrodes. Thus the system of the invention preferably comprises an electrode in contact with the novel protein. This offers potentiostatic control over the redox state of the surface layer, and the possibility to perform scanning voltammetry while detecting the fluorescence intensity as a monitor of the redox state of the surface-bound proteins.

The method may be performed in an optical set-up that makes use of total internal reflection to excite a layer of fluorescently labelled protein molecules. The electrodes are mounted in an optical microscope equipped with laser excitation and a high aperture objective to monitor the fluorescence emitted from the protein coated on the electrode. The electrodes are transparent to light of wavelength for exciting the fluorescent label and to the fluorescence emitted by the label. In addition, a three electrode electrochemical set-up may be connected to the sample compartment and the electrode immersed in buffer to which enzyme substrate can be added. The enzyme may be regenerated either by a voltage sweep or chemically by making the electrode part of the flow cell and directing a redox active flow over the electrode.

The system may be used in a biosensor to monitor the activity of redox enzymes and proteins with a greater sensitivity than in conventional methods. Experiments in the lower picomolar range are within reach, which opens up opportunities for investigating molecules which are only available in minute quantities. Since Cy5 is a common dye for single-molecule fluorescence detection the method presented here has the potential to study redox events in enzymes and proteins at the single-molecule level. This greater sensitivity leads to specific advantages: almost unlimited miniaturization, applicability to much lower concentrations (sub-nanomol/L) and strongly enhanced specificity due to the absence of interference. The proposed system has great potential for application in high-throughput screening and in nanotech-based bioelectronics.

### BRIEF DESCRIPTION OF THE DRAWINGS

FIG. 1: Method of covalently linking the dye to a cysteine through oxidative coupling with a maleimide derivative of Cy5.

FIG. 2: Room temperature absorption (black) and emission spectrum (grey) of dye Cy5, and absorption spectrum of oxidized azurin (dashed).

FIG. 3: Ribbon representation of azurin structure showing the positions of engineered cysteines. Gln12Cys is abbreviated to Q12C, Lys27Cys to K27C and Asn42Cys to N42C.

FIG. 4: Fluorescence intensity of a solution of amicyanin, methylamine and MADH (points of addition are indicated by arrows.)

FIG. 5: Room temperature fluorescence intensity, vertical scale (arbitrary units) as a function of time (secs).

A: Solution containing Cy5 labelled N42C. At time (t)=90, 480, 790 s (arrows labelled 1, 3 and 5) aliquots of DTT in water are added. At t=400 and 625 s (arrows labelled 2 and 4) aliquots of K₃Fe(CN)₆ in water are added.

B: Similar experiment on the Zn form of the Cy5 labelled N42C azurin variant (lower trace) and a solution containing Cy5 only (upper trace).

FIG. 6: Azurin absorption spectra (A) and estimated resonance energy transfer efficiency between Cy5 and the oxidised type-1 Cu site of azurin (B). In a A solid line=spectrum of oxidised azurin, dotted line=reduced azurin and dashed line=fluorescence spectrum of Cy5. In B, solid vertical line=estimated donor-acceptor distance and dotted vertical lines its estimated error.

FIG. 7: Changes in fluorescence intensity of blue copper proteins with Cy5-labelled N-terminus upon oxidation and reduction. A=azurin with Cu replaced by Zn; B=azurin, C=amicyanin, D=plastocyanin, E=pseudoazurin. Arrows indicate addition of excess oxidant (1) or reductant (2).

FIG. 8: Potentiometric titrations of azurin by absorption and fluorescence. Squares=intensity of the fluorescence of Cy5 attached to the protein at 665 nm, Solid line=Nernst fit of fluorescence intensity, Circles=azurin absorption at 630 nm, dashed line=Nernst fit of absorption.

FIG. 9: Cytochrome c550 absorption spectra (A) and estimated resonance energy transfer efficiency between Cy5 and the heme of the cytochrome (B). In A, solid line=oxidised cytochrome, dotted line=fluorescence spectrum of Cy5. In B, thick line=oxidised cytochrome, thin line=reduced, vertical line its estimated error.

FIG. 10: Potentiometric titrations of cytochrome c550 by absorption and fluorescence. Squares=intensity of the fluorescence of Cy5 attached to the protein at 665 nm, solid line=Nernst fit of fluorescence intensity, circles=cytochrome absorption at 550 nm, dashed line=Nernst fit of absorption.

FIG. 11: Flavodoxin II absorption spectra (A) and estimated resonance energy transfer efficiency between Cy5 and the flavin of the flavodoxin. In A, solid line=fully oxidised flavodoxin, dotted line=singly reduced (semiquinone) flaxodoxin and dashed line=fluorescence spectrum of Cy5. In B, thick line=oxidised flavin, thin line=semiquinone. Vertical lines as in FIG. 6.

FIG. 12: Potentiometric titrations of flavodoxin II by absorption and fluorescence. Squares=intensity of the fluorescence of Cy5 attached to the protein at 665 nm, solid line=Nernst fit of fluorescence intensity, circles=flavodoxin absorption at 577 nm, dashed line=Nernst fit of absorption.

FIG. 13: Time course of Cy-5 labelled MADH upon MA addition.

FIG. 14: Kinetic traces obtained from labelled NiR upon reduction with various concentrations of sodiumdithionite (DT).

FIG. 15: Kinetic traces from redox “inactive” labelled NiR upon reduction.

FIG. 16: Time course of Cy5 labelled NiR upon reduction, nitrite conversion and complete oxidation.

FIG. 17: Optical absorption spectra of wild-type SLAC. Solid line=fully oxidised SLAC, grey line=reduced SLAC, broken line=oxidised laccase from which Type-1 Cu site had been deleted by site-directed mutagenesis.

FIG. 18=Endogenous SLAC tryptophan fluorescence. A=fluorescence emission spectra of wt SLAC in reduced form (black line) and oxidised from (grey line). B=Decrease in Trp emission intensity when reduced SLAC is mixed with O₂. C=Rate of oxygenation as determined by stopped-flow fluorescence spectroscopy.

FIG. 19: Emission spectra of 1 μM labelled laccase in the reduced (black line) and oxidised (grey line) state.

FIG. 20: reduction of SLAC by dithionite under anaerobic conditions at pH 6.8 and pH 9.5. Lines labelled T4=endogenous Trp fluorescence, lines labelled T1=Cy5 emission. The schematic indicates the reduction events taking place.

FIG. 21: Approach to the steady-state in SLAC catalysed turnover of 2,6-dimethoxyphenol. T4=Trp fluorescence, T1=Cy5 fluorescence and on the right graph product absorption at 468 nm. Reaction scheme shown schematically in upper part of figure.

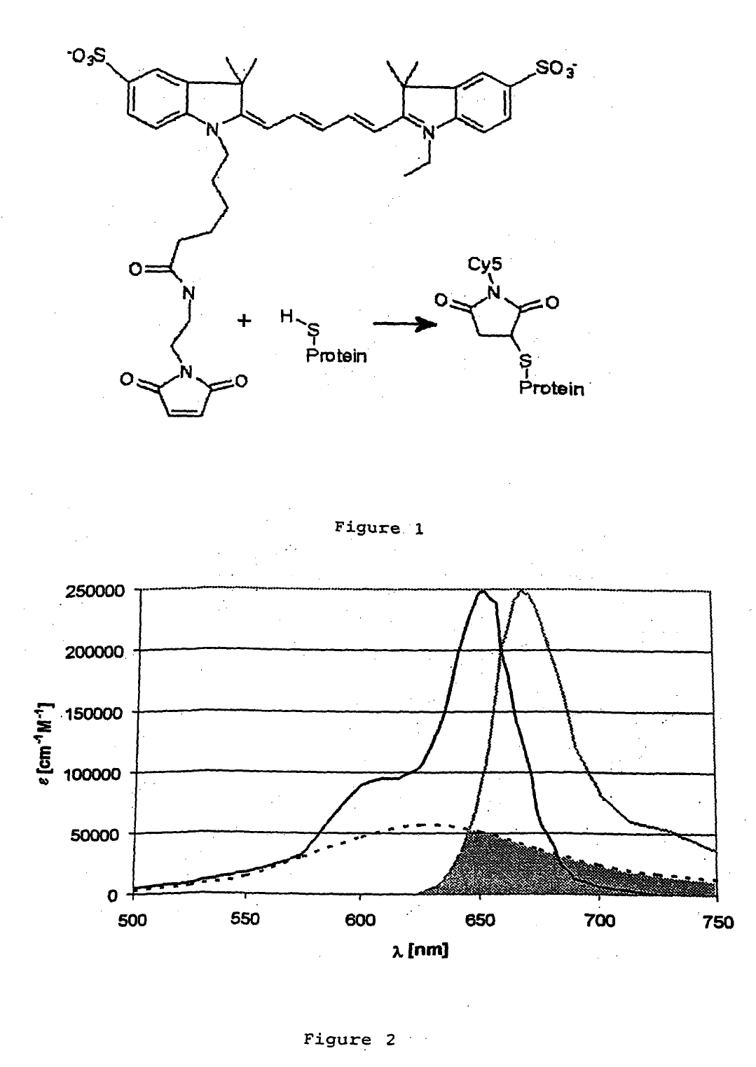

The invention may be exemplified by the following worked examples:

### EXAMPLE 1

The absorption and fluorescence spectra of Cy5 and the absorption spectrum of oxidised azurin were measured. Fluorescence was measured with a LS 5OB or LS55 commercial fluorimeter (Perkin Elmer, USA), With a red sensitive photomultiplier (R928, Hamamatsu, Japan), set to 5 nm band pass.

FIG. 2 shows the room temperature absorption (black) and emission spectrum (grey) of Cy5, and absorption spectrum of oxidised azurin (dashed). The vertical scale for the extinction corresponds with the absorption spectra and the vertical scale for the emission spectrum is in arbitrary units. The azurin spectrum has been expanded in the vertical direction by a factor of 10. The region of spectral overlap between the donor emission fluorescence and the acceptor absorption is indicated by the grey area.

### EXAMPLE 2

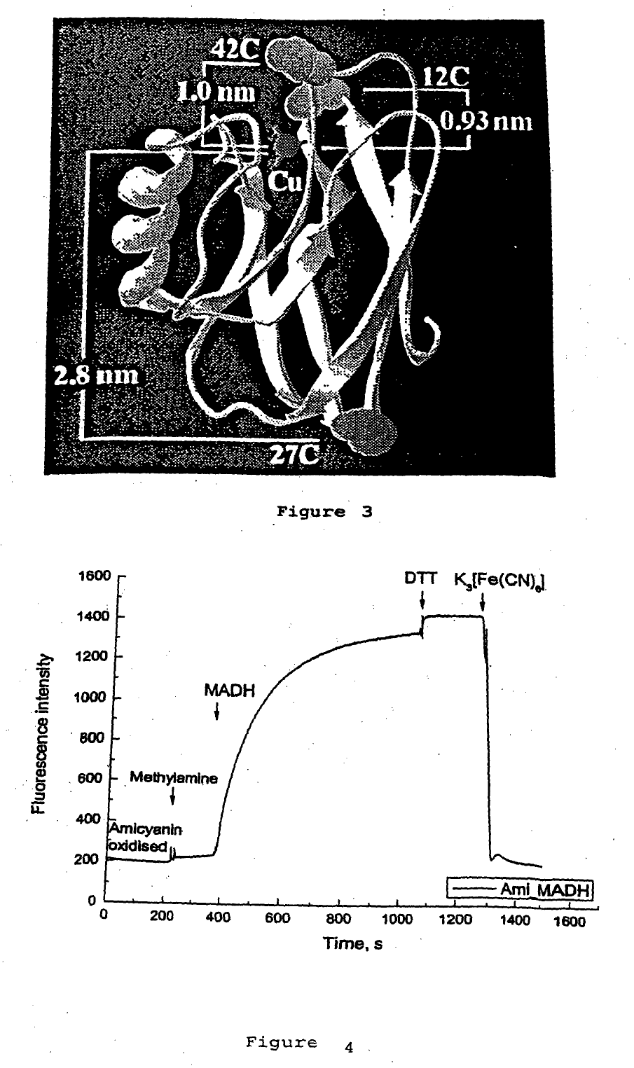

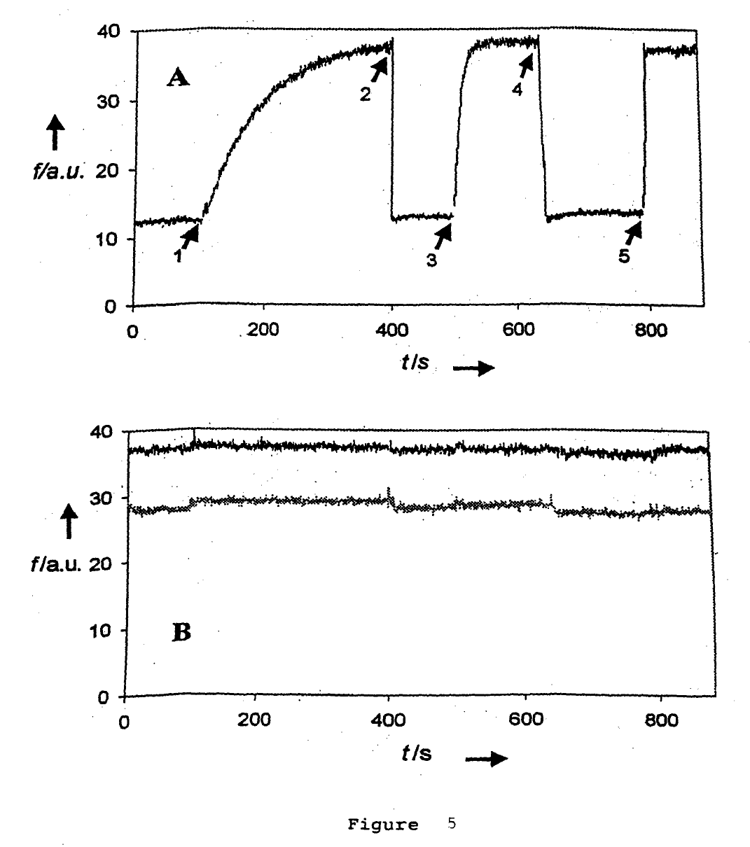

For site-specific fluorescent labelling with the dye Cy5 (ε=250 mM⁻¹ cm⁻¹), three cysteine mutants of azurin, Q12C, K27C and N42C, with cysteines at positions 12, 27 or 42 in the amino acid chain, respectively were prepared. The cysteines were all at different distances from the copper site (as measured from the C_{α} carbon atom), as shown in FIG. 3. Co-ordinates were taken from the Protein Database (4AZU & 5AZU ) [10]. Note that the length of the amino acid side chain, the spacer length and the dye size (totaling ˜1 nm) still have to be added to obtain the distance between Cy5 and the Cu atom. Preparation and purification of the mutants N42C and K27C was carried out according to published procedures [11].

Holo-azurin (i.e. azurin containing copper) is obtained after the expression of the azu gene in E. coli. Apo-azurin (i.e. protein which lacks any metal in the active site) may be obtained as follows [12]:

### Preparation of Apo-Azurin

100 ml of a 0.1 M KCN solution in 0.15 M Tris/HCl (pH 8.3) containing the required amount of reduced (reduction by dithionite) holo-azurin is stirred overnight at 4° C. Cyanide is removed by ultrafiltration and the produced apo-azurin is transferred to the required buffer by repeated concentration and dilution. This results in an apo-azurin preparation with better than 95% purity.

Zinc azurin may then be prepared from apo-azurin as follows: [13]

### Preparation of Zinc Azurin

A 20 micro M solution of apo-azurin in 50 mM ammoniumacetate (pH 6.0) is incubated with excess Zn-chloride (10-100 equivalents) at 37° C. for a few hours. This results in virtually quantitative conversion of the apo-form into the metal containing azurin. The protein is then purified by column chromatography.

The labelling of the azurin with the dye Cy5 was carried out as follows:

Cy5 maleimide (from Amersham Biosciences; Freiburg, Germany) was dissolved in water free dimethylsulfoxide (DMSO) to a concentration of roughly 30 mM. All purification steps were performed using centri-spin 10 size-exclusion chromatography spin columns with a 5 kDa cut off (Princeton Separations; Adelphia, N.J., USA) according to the manufacturer's instructions. Labelling of K27C form. Apo-protein solution (˜16 μM) was incubated at room temperature for 1 h with 3 mM dithiothreitol (DTT). This step was necessary to break up dimers which might have formed via the introduced cysteine [13].

Removal of DTT and buffer exchange to 20 mM Tris pH 7.0, 100 mM NaCl was achieved by size exclusion chromatography, after which the sample was incubated with 1 mM Tris(2-carboxyethyl)-phosphine hydrochloride (TCEP) to minimize the formation of disulfide bridges. Subsequently CuNO₃ was added up to 50 μM (roughly 4 times molar excess over azurin). After 10 minutes at room temperature Cy5 was added up to 10 times molar excess. After 1 h free dye was removed by two consecutive size exclusion chromatography steps and the sample was transferred into phosphate buffered saline (PBS) solution (150 mM NaCl, 10 mM Na₂HPO₄ NaH₂PO₄, pH 7.4), which is known for its low fluorescent background.

Labelling of Q12C and N42C. The copper form of the protein was treated with 1 mM DTT to remove potential dimers as described above, and transferred into labelling buffer. After 20 minutes a 5 times excess of Cy5 maleimide was added. After 1 h incubation at room temperature the labelled protein was purified as above.

All zinc forms were directly transferred into PBS and a 5-10 fold excess of Cy5 maleimide was added. After one hour the protein was purified in a manner similar to the copper form.

### Fluorescence Quenching Observation

The protein-dye constructs were used to investigate the influence of the redox state of azurin on the fluorescence of the attached fluorophore. Cy5 labelled azurin was either oxidized by adding K₃Fe(CN)₆ or reduced by adding DTT, and the fluorescence was recorded as a function of time. The experiment was carried out as follows:

### Part A

A solution containing 3.4 nM of Cy5 labelled N42C Cu azurin in 1:10 diluted PBS buffer pH 7.4 was prepared. At the start of the experiment (t=0) the protein is in the oxidized form. At t=90, 480 and 790 s (arrows labelled 1, 3 and 5 in FIG. 5a) aliquots of a 100 mM solution of DTT (an oxidant) in water were added. The summed concentrations of added reactant after the additions amounted to 1.5, 6 and 14.5 mM, respectively. At t=400 and 625 s (arrows labelled 2 and 4) aliquots of a 100 mM solution of K₃Fe(CN)₆ in water were added. The summed concentrations of this reactant after the additions amounted to 2.5 and 7.4 mM, respectively. The excitation wave length was 645 nm and emission was collected at 665 nm.

### Part B

A similar experiment was performed on a 1.1 nM solution of the Zn form of the Cy5 labelled N42C azurin variant (lower trace in FIG. 5B) and a solution containing 1.4 nM of Cy5, only (upper trace). The buffer consisted of 1:10 diluted PBS at pH 7.4. Additions of reductant and oxidant were made at approximately the same time points as in part A. The small stepwise intensity changes observed for the lower trace are due to a slight contamination of the Zn azurin with the Cu form and slight dilution effects.

The results are shown in FIG. 5. At t=0 (see FIG. 5A) the azurin is in the oxidised form. Addition of an excess of DTT at t=100 s causes a threefold increase in the fluorescence while subsequent addition of an excess of the oxidant brings the fluorescence back to the original level. Further additions of reductant and oxidant show that the switching is reversible. It is clear that in the beginning (100-400 s) the reduction rate is much slower than the oxidation rate (at t=400 s), but that the reduction becomes faster at later stages. This is because the increasing amount of ferri/ferrocyanide, added at subsequent oxidation steps, acts as a mediator for the reductant.

The experiments with Cy5 labelled redox inactive zinc azurin and with a solution containing only the Cy5 label acted as controls. The results are shown in FIG. 5B. It is clear that the switching of the fluorescence as observed in FIG. 5A is absent.

The assumed quenching mechanism, fluorescence resonance energy transfer (FRET), is a strongly distance dependent process. The energy transfer efficiency, E, as a function of distance R between the Cy5 label and the Cu site is given by equation 2 [8]:

$$E = \frac{{F}_{o} - {F}_{r}}{{F}_{o}} = \frac{{R}_{o}^{6}}{{R}_{o}^{6} + {R}^{6}} \qquad Equation 2$$

Fᵣ and Fₒ denote the fluorescence intensity of the labelled azurin in the reduced and oxidised form respectively. Rₒ is a characteristic distance that depends [7] on the refractive index, n, the spectral overlap between donor and acceptor bands, J(λ), the fluorescence quantum yield of the donor, Q_{D}, and the relative orientation of the optical transition moments of donor (Cy5) and acceptor (Cu center) as reflected by the orientation factor k². The latter may vary between 0 and 4 and amounts to \ddot{2}\ddot{/}\ddot{3} for two freely rotating dipoles.

With Q_{D}=0.27, [14], k²=⅔, n=1.4 and J(λ)=7.3×10⁻¹⁴ M⁻¹ cm³ we obtain a Förster radius R₀ of 3.8 nm for oxidised azurin. The actual value of R₀ may differ by as much as 20-30% from this value depending on k² and the conformation of the label with respect to the protein. The purpose of the calculation is not to obtain a precise value of R₀, but to show that for the combination of donor and acceptor chosen here, R₀ is of a similar size as azurin, which has dimensions of 2.5×3'4 nm. Thus, a bound Cy5 label will exhibit a sizeable quenching rate in the oxidised azurin while quenching is absent in the reduced protein in agreement with the experimentally observed effects (FIG. 5). For the other two azurin variants (K27C and Q12C) similar results were obtained. The data are summarized in Table 2.

TABLE 2
Quenching rates observed for the three Cy5 labelled azurin variants
listed in the first column and calculated distances between the Cu
and the C_{α} positions of the engineered Cys residues.
Distances were taken from pdb-files 4AZU & 5AZU [10].

| Azurin Variant | C_{α}-Cᵤ distance, nm | Quenching rate |
|---|---|---|
| Q12C | 0.9 | 0.570 |
| K27C | 2.8 | 0.540 |
| N42C | 1.0 | 0.781 |

### EXAMPLE 3

The reaction between two partner proteins, Methylamine dehydrogenase (MADH) and amicyanin, was followed by monitoring the fluorescence quenching of the label on amicyanin.

### (i) Labelling

1.5 μL of aminoreactive Cy5 dye was added to 50 μL of oxidised wt amicyanin (100 μM) in HEPES pH 8.3 and incubated for 2 h. at room temperature. The unbound dye was washed out by 2 subsequent centrifugations on Centrispin 10 columns (3 minutes at 3000 rpm).

### (ii) Fluorescence Measurements

Fluorescence was measured on a Perkin-Elmer fluorimeter in a quarz cuvette with 5 mm pathlength. The dye was excited at 645 nm and the fluorescence was monitored at 665 nm. At t=0 oxidised labelled amicyanin was added to the cuvette to a final concentration of 0.25 μM. Then 10 mM methylamine (the substrate) was added to the sample and finally 0.7 μM of oxidised wt MADH. Excess DTT was added to check whether amicyanin was fully reduced and excess K₃[Fe(CN)₆] was added at the end of the experiment to re-oxidise the amicyanin, bringing the fluorescence intensity back to base level.

The scheme of the reactions taking place in the cuvette is as follows: MADH+H₃CNH₂+H₂O→MADH⁻+H₂CO+NH₄⁺+H⁺; 2Ami+MADH⁻→2Ami⁻+MADH.

The points of additions are indicated with arrows in FIG. 4. Amicyanin is oxidised (contains Cu²⁺) at the start of the experiment and the fluorescence intensity is low. This is because Cu²⁺ is able to quench the dye's fluorescence. As soon as substrate methylamine and partner protein MADH are added the fluorescence intensity starts to increase as the amicyanin is reduced (Cu²⁺→Cu⁺) and the copper ion is no longer able to quench the dye's fluorescence.

In summary, examples 1-3 demonstrate that the fluorescence of a dye coupled to a protein can be strongly affected by a change in oxidation state of the protein. This documents a very sensitive way to monitor changes in the redox state of a protein. The protein concentrations used in example 2 amount to a few nM. Considering the signal to noise (S/N) ratio observed in FIG. 5A, the concentrations can be easily lowered by two or more orders of magnitude without decreasing the S/N ratio to an unacceptable level even more so when signal-averaging techniques are employed.

### EXAMPLE 4

The following example demonstrates a method for fluorescence detection of protein redox state based on resonance transfer to three types of prosthetic groups: pseudo-azurin, amicyanin, plastocyanin and azurin (all containing a type-1 Cu site), a hemoprotein cytochrome c550 and a flavin mononucleotide-containing flavadoxin.

### Materials

Wild type azurin from Pseudomonas aeruginosa was overexpressed in E. coli and purified as previously described [12]. Cytochrome c550 from Paracoccus versutus was expressed and purified as earlier described [15]. Flavodoxin II C69A/V100C from Azotobacter vinelandii ATCC 478 was purified as described previously [16]. Amicyanin from Paracoccus versutus, plastocyanin from Dryopteris crassirhizoma and Alcaligenes faecalis pseudoazurin were expressed and purified as described elsewhere [17-19].

Cy5 maleimide and NHS-ester were purchased from Amersham Biosciences (Freiburg, Germany). The stock solutions of the dyes were prepared by dissolving them in water-free dimethylsulfoxide to a concentration of roughly 30 mM. All purification steps during protein labeling were performed using Centrispin 10 size-exclusion chromatography spin columns with a 5 kDa cutoff (Princeton Separations; Adelphia, N.J., USA) according to the manufacturer's instructions.

### Protein Labeling

Flavodoxin II C69A/V100C was labeled on the mutated cysteine residue (Cys100) with Cy5 maleimide, whereas all other proteins were labeled at amino groups using Cy5 NHS-ester.

For amino labeling, Cy5 NHS-ester was added in 10 times molar excess to the 100 μM proteins in HEPES 20 mM, pH 8.3 and incubated for 2 hours at room temperature. These conditions are recommended by the manufacturers for N-terminal labeling. The unbound label was then removed by two consecutive size-exclusion chromatography steps.

For cysteine labeling, 100 μM C69A/V100C flavodoxin in HEPES 20 mM pH 7.0 was first incubated with 10 times molar excess of dithiotreitol (DTT) for 1 hour at room temperature to break the possible disulfide bridges between the introduced cysteines. After incubation, excess dithiotreitol was removed by a single step of size-exclusion chromatography. Then Cy5 maleimide was added to the protein in about 10-fold molar excess and left for 1 hour at room temperature before removing the unbound label as above.

The protein labeling ratio (dye/protein molecule) was estimated from the absorption spectra of labeled proteins, using ε₆₄₅=250 mM⁻¹ cm⁻¹ for Cy5 [21], ε₂₈₀=9.8 mM⁻¹ cm⁻¹ for azurin [22], ε₄₁₀=134 mM⁻¹ cm⁻¹ for cytochrome c550 [23] and ε₄₅₂=11.3 mM⁻¹ cm⁻¹ for flavodoxin [17].

### Fluorescence And Absorption Spectroscopy

Absorption spectra were measured using a Perkin Elmer Instruments Lambda 800 spectrophotometer with a slit width equivalent to a bandwidth of 2 nm. Fluorescence spectra and time courses were measured with an LS 55 commercial fluorimeter (Perkin Elmer, USA), with a red sensitive photomultiplier (R928, Hamamatsu, Japan), set to 8 nm band pass. Cy5 fluorescence was excited at 645 nm, fluorescence intensity at 665 nm was used for the analysis of the FRET efficiency.

### Fluorescence Time Courses

Fluorescence time courses were measured in a 5×5 mm quartz fluorescence cuvette (Perkin Elmer) in 20 mM HEPES, pH 7 or pH 8.3. The protein concentration was 1-10 μM. Protein reduction and oxidation during measurement was performed by adding reductants (dithiotreitol or ascorbate) and oxidant (sodium ferricyanide) from concentrated stock solutions directly into the cuvette to a final concentration of 1-3 mM.

### Redox Titrations

Potentiometric redox titrations were performed in 20 mM HEPES, pH 7 or pH 8.3 using a home made spectrophotometric cuvette for potentiometric titrations as described by Dutton [23] with 10 mm optical pathlength. A saturated calomel electrode was used as a reference electrode. A gold rod electrode (BAS Electrochemistry) was used as a measuring electrode for azurin and cytochrome titrations. For the C69A/V100C flavodoxin titration we used a platinum measuring electrode to avoid possible interaction of the surface cysteine with the gold electrode. Potassium ferricyanide and dithiotreitol (azurin and cytochrome) or sodium dithionite (flavodoxin) were used to change the potential of the solution. When dithionite was used as a reductant, the buffer was deoxygenated in the potentiometric cuvette prior to measurements by passing Ar through it for 3 hours. After that the protein was added and deoxygenation was continued for 30 minutes. An Ar flow over the sample was also maintained during the measurements. In the flavodoxin titration 12 μM benzylviologen was added to the sample at the start of the titration as a mediator to facilitate protein reduction by sodium dithionite.

### Förster Radius Calculations

R₀ was calculated as previously described [8] from the equation R₀=0.211(Jk²n⁻⁴Φ_{D})^{1/6} (Å). Here k² is an orientation factor, n—refractive index, Φ_{D}—fluorescence quantum yield of the donor and J—spectral overlap integral, defined as J=∫(F_{D}(λ)ε_{A}(λ)λ⁴/∫F_{D}(λ)dλ where F_{D}(λ)is the fluorescence intensity of the donor, ε_{A}(λ)—the extinction coefficient of the acceptor at wavelength λ with λ expressed in nanometers. Experimental protein absorption spectra and the Cy5 fluorescence spectrum supplied by the manufacturer (Amersham Biosciences) were used for the calculations. The refractive index was assumed to be 1.4 and the orientation factor k² was taken to be ⅔ which corresponds to random orientations of both donor and acceptor [8]. Φ_{D} for Cy5 was taken to be 0.27 [14].

The distance (R) from Cy5 to the accepting prosthetic group was estimated as R=(d+1) nm±0.5 nm, where d is the distance from the attachment point of the dye (N-terminus for azurin, lysines for cytochrome and Cₐ of the Cys100 for the C69A/V100C flavodoxin). The distance d was estimated from the protein crystal structures. Adding 1 nm to the calculated distance d accounts for the approximate length of the linker chain.

### Mass Spectrometry

Electrospray ionization (ESI) mass spectrometry analyses of the intact protein were carried out on a MicroTOF instrument (Bruker Daltonics, Bremen). Protein samples (5-10 pmol/μl) dissolved in 0.2% formic acid and 50% methanol were continuously infused into the ESI source at a flow rate of 180 μl/hour. Spectra were recorded in the positive ion mode and the standard m/z range of 200-3000 was monitored. Molecular masses of proteins were calculated using a maximum entropy deconvolution algorithm incorporated as part of the DataAnalysis software supplied with the mass spectrometer.

For matrix-assisted laser desorption (MALDI) analysis of the trypsin-digested labeled proteins about 100 pM protein was resuspended in 100 μL of 25 mM ammonium bicarbonate (pH=8.0). To this 2 μL of trypsin (1 μg/μL) solution was added. The reaction was carried out at 37° C. for 16 h. After digestion, the peptides were desalted using Poros 50 R2, packed in a pipette tip. Peptides were eluted in 60% acetonitrile/0.01% TFA and measured by MALDI-MS (Ultraflex II, Bruker Daltonics, Bremen) using α-cyano-4-hydroxycinnamic acid as a matrix.

### Results And Discussion

### Optimization of the Labeling Conditions

The labeling conditions were optimized to ensure that the dye-to-protein ratio was less than one.

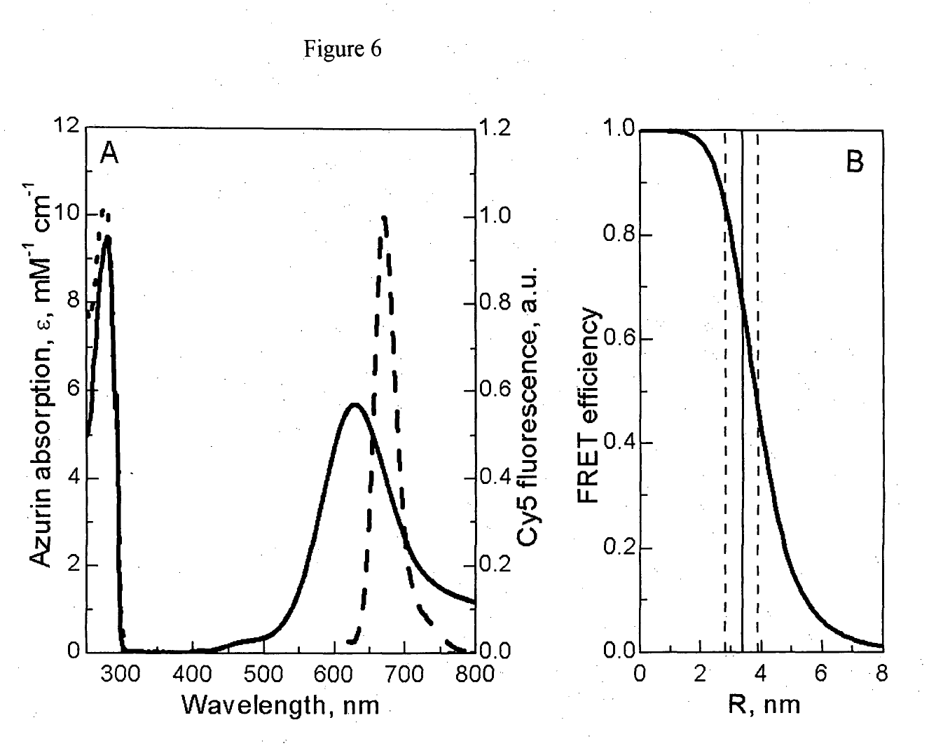

For azurin and cytochrome c550 electrospray ionization mass spectrometry were performed to check the number of label molecules per protein. For C69A/V100C flavodoxin mutant this was deemed unnecessary since there is only a single cysteine available for the Cy5-maleimide binding. For azurin and cytochrome c550 only peaks arising from unlabeled and singly labeled proteins were observed, showing that no protein molecules with multiple labels were present in the sample (see FIG. 6 for azurin, for the cytochrome c550 the results are not shown).

### a) Fluorescence Determination of Protein Redox States: Azurin And Other Blue Copper Proteins

Azurin from Pseudomonas aeruginosa is a small (14 kDa) electron transfer protein containing a type-1 Cu centre.

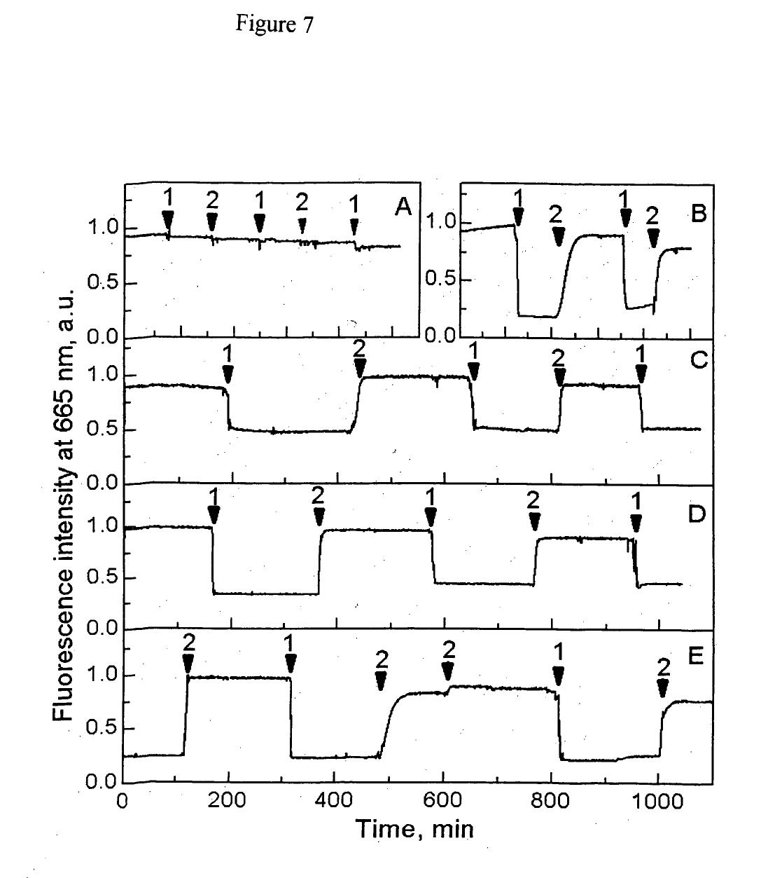

The absorption band at 590-630 nm present in the Cu(II) state and absent in the Cu(I) state is a common feature for all the type-1 Cu centres. It can, thus, be expected that other blue copper proteins, labeled with Cy5, will also show a significant resonance energy transfer from the fluorophore to the Cu centre in the oxidized but not in the reduced state. FIG. 7 shows the changes in fluorescence intensity of several blue copper proteins with a Cy5-labeled N-terminus upon oxidation and reduction. Pseudomonas aeruginosa azurin, amicyanin from Paracoccus versutus, plastocyanin from Dryopteris crassirhizoma and Alcaligenes faecalis pseudoazurin all show a significant decrease in fluorescence intensity upon oxidation, while on protein reduction fluorescence goes back to almost the initial value (FIGS. 7B, C, D and E). The effect does not depend on whether at the start of the experiment the protein is oxidized (FIG. 7E) or reduced (FIG. 7B, C, D). As all the studied blue copper proteins have similar absorption spectra as well as similar shape and size (9-15 kDa), the Förster radii for Cy5-type-1 Cu(II) resonance energy transfer and the donor-acceptor distances are also expected to be similar. The data in FIG. 7 show that azurin, amicyanin, plastocyanin and pseudoazurin labeled on the N-terminus with Cy5 are about 80% less fluorescent in the oxidized than in the reduced state. This value is also in good agreement with the expected energy transfer efficiency of 65±20% from Cy5 attached to the N-terminus of the type-1 Cu centre for oxidized azurin (FIG. 7B).

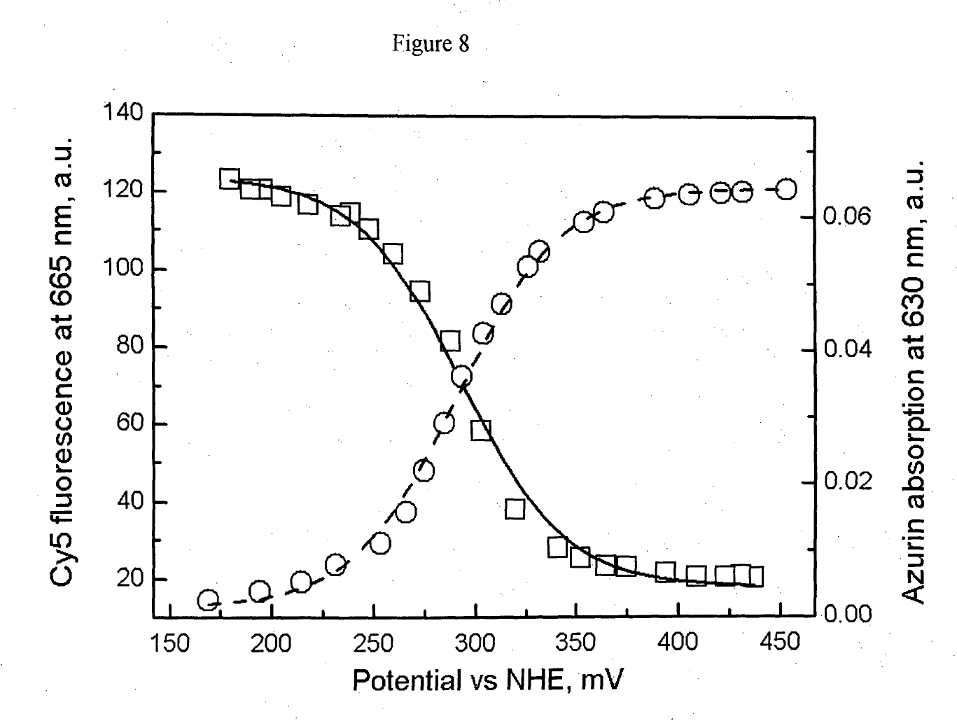

A potentiometric titration of azurin labeled with Cy5 on the N-terminus has been performed. FIG. 8 shows a potentiometric titration of azurin monitored by the absorption at 630 nm and the titration of azurin labeled on the N-terminus with Cy5, monitored by Cy5 fluorescence at 665 nm. It can be seen that the fluorescence intensity of the attached dye goes up as the absorption of the type-1 Cu(II) site at 630 nm decreases. The midpoint potentials obtained from the fits of both titration curves to the Nernst equation coincide (293±2 mV vs NHE for the fluorescence and 291±2 mV vs NHE for the absorption titration) and are in good agreement with the previously reported value of 292 mV. vs NHE for the midpoint potential of Pseudomonas aeruginosa at pH 8 [27]. It should be noticed that the protein concentration used for the fluorescence titration is 40 times smaller than the one used for the absorption titration. It shows that the fluorescence method for monitoring the protein redox state has a significantly higher sensitivity compared to the absorption method.

### b) Fluorescence Determination of Protein Redox States: Cytochrome c550

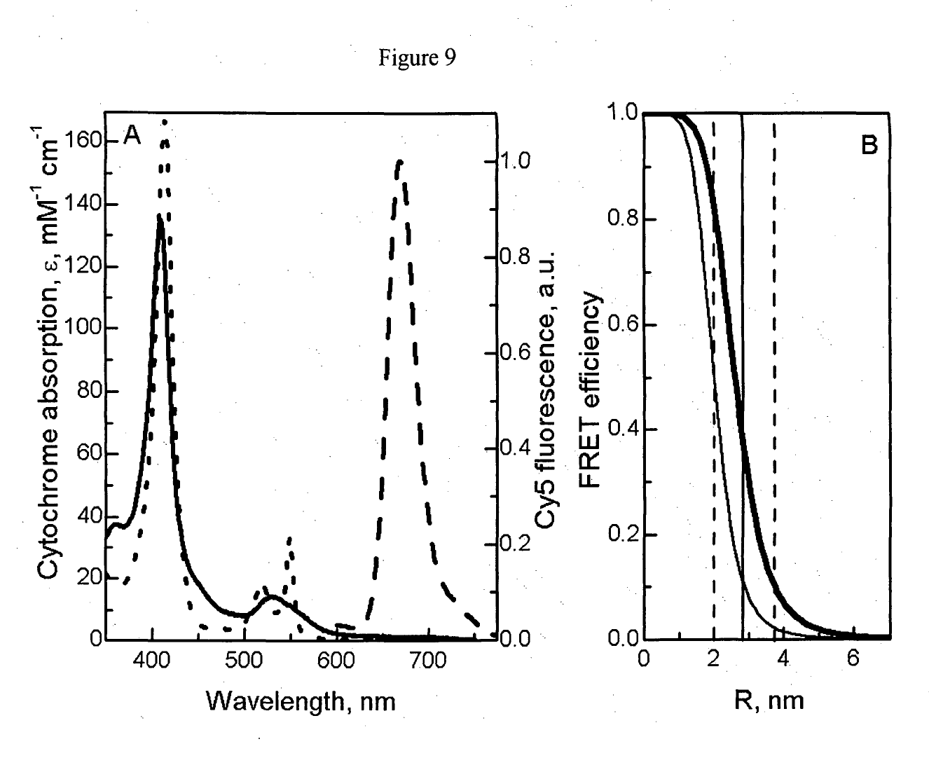

Cytochrome c550 from Paracoccus versutus is a 14.7 kDa heme-containing electron carrier protein present in the methylamine oxidising chain of this bacterium where it acts as an electron donor for the membrane-bound cytochrome c oxidase [22]. It belongs to the class I of c-type cytochromes and contains a covalently-bound heme located asymmetrically near the protein surface, which is low-spin both in the oxidized and reduced forms. Reduced cytochrome c550 shows an intense absorption band at 416 nm (Soret band), a sharp peak at 550 nm (a band) and a smaller band at 522 nm (b band). In the oxidized form of the protein the Soret band is shifted and decreases in intensity, while a and b bands merge into a single broad absorption peak. Oxidized cytochrome also absorbs in the region of 570-750 nm, where the absorption of reduced cytochrome is significantly lower (FIG. 9A). We chose Cy5 as a fluorescent donor, as the overlap between its fluorescence and the cytochrome absorption shows a small but stable increase on cytochrome oxidation (FIG. 9A). The estimated Förster radii for FRET from Cy5 to the heme are 2.6 nm for the oxidized and 2.0 nm for the reduced cytochrome. (FIG. 9B). As the MALDI analysis of the labeled cytochrome c550 after trypsinolysis indicates that Cy5 has an equal probability to attach to any of the exposed lysines, the donor-acceptor distance from Cy5 to the heme is estimated from the crystal structure [25] as an average over all the possible attachment points and equals 2.8±0.8 nm. For this donor-acceptor distance the estimated difference between the maximal and minimum fluorescence is about 30% (FIG. 9B).

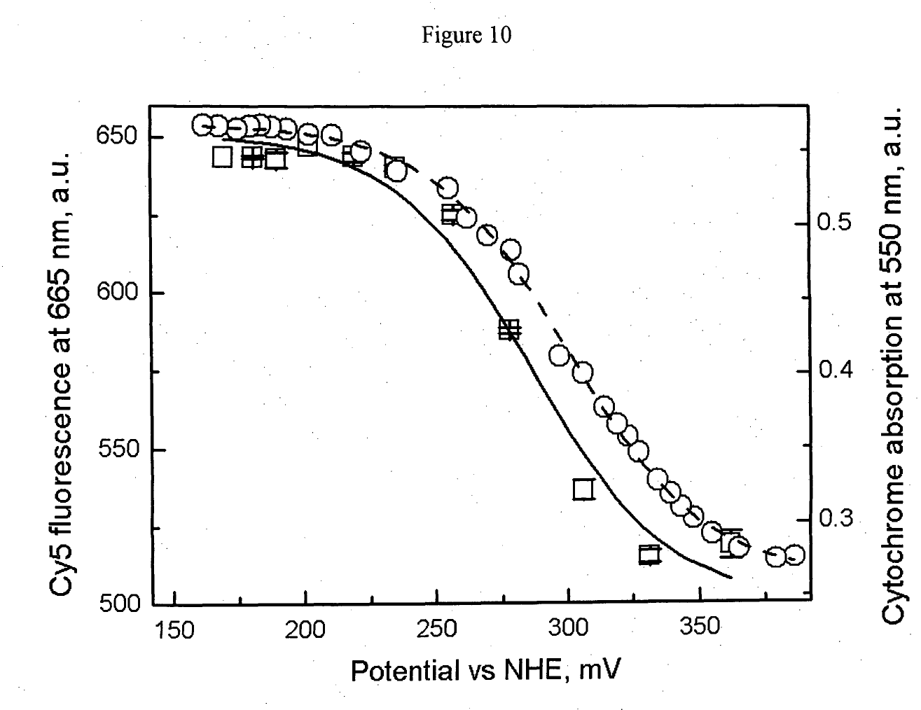

FIG. 10 shows potentiometric titrations of cytochrome c550 based on the absorption at 550 nm and of cytochrome labeled with Cy5 NHS-ester based on the fluorescence at 665 nm. The Nernst fit of the absorption titration gives a midpoint potential of 300±1 mV vs NHE, the fit of the titration by fluorescence gives a midpoint of 286±4 mV vs NHE. The small discrepancy between the two values may be due to small variations between the lowest and highest fluorescence intensities leading to imprecise measurement of the midpoint potential on the basis of fluorescence. Both values for the midpoint potentials observed in this study are slightly higher than the previously reported value of 255 mV vs NHE [21].

### c) Fluorescence Determination of Protein Redox States: Flavodoxin

Flavodoxins are electron transfer proteins, containing flavin mononucleotide (FMN) as a prosthetic group. FMN can exist in three possible redox states: oxidized (quinone), one-electron reduced (semiquinone) and two-electron reduced (hydroquinone). While in most cases flavodoxin expression is induced by iron deficiency, in Azotobacter vinelandii flavodoxin is expressed constitutively [26] and is likely to be an electron donor for the nitrogenase [9]. Azotobacter vinelandii flavodoxins were reported to be unusually stable in the semiquinone form compared to other flavodoxins [17; 27], facilitating the study of the one-electron reduced state of this protein.

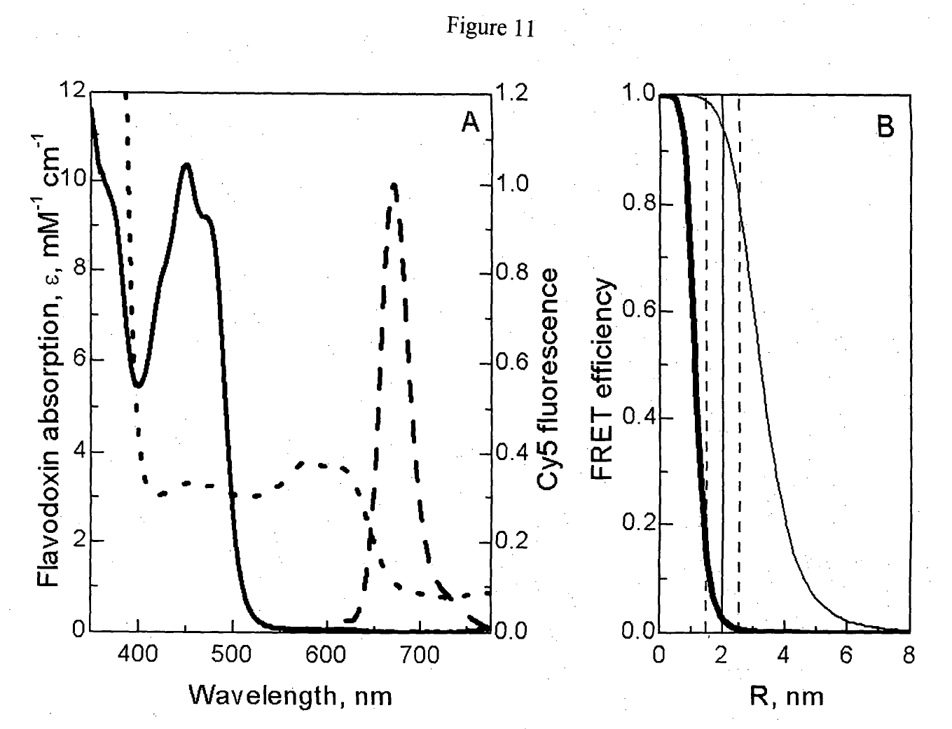

FIG. 11A shows the absorption spectra of oxidized and singly reduced flavodoxin II from Azotobacter vinelandii ATCC 478. In the semiquinone state a broad absorption peak appears between 580 and 620 nm that. extends to 700 nm, which is not present in either the fully oxidized or the fully reduced state while the quinone form still has a weak absorption above 550 nm (FIG. 11A). This makes Cy5 a suitable donor to distinguish between the oxidized and one-electron reduced flavodoxin using FRET efficiency (FIG. 11B). The estimated Förster radii for FRET from Cy5 are 3.2 nm for the one-electron reduced flavodoxin and 1.1 nm for the fully oxidized state. We used the C69A/V100C flavodoxin mutant, in which the natural exposed Cys69 is replaced by Ala and a new cysteine is introduced in position 100. Cys100 is only 9 Å from the flavin [28] and thus the donor-acceptor distance for the Cy5 attached to Cys100 can be roughly estimated as 2±0.5 nm.

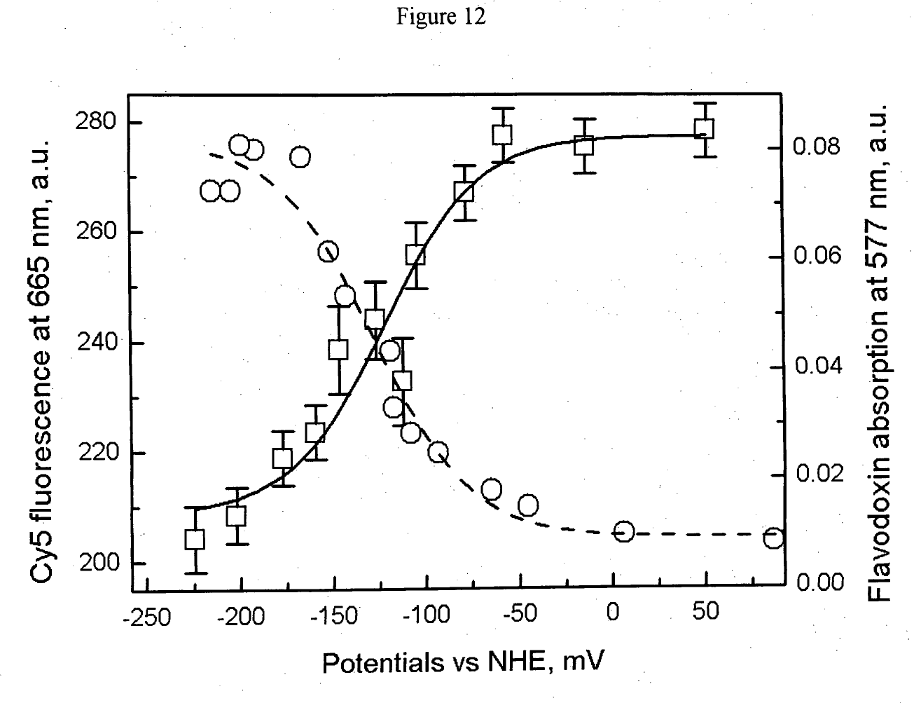

Potentiometric titrations of C69A/V100C flavodoxin by absorption at 577 nm and fluorescence of the Cy5 label attached to Cys100 at pH 7 are shown in FIG. 12. The data show that the fluorescence intensity decreases as the absorption at 580-620 nm goes up. The Nernst fits of absorption and fluorescence titrations give identical midpoint potentials (−120±9 mV vs NHE for the fluorescence and −126±5 mV vs NHE for absorption). This value is in the interval of −45±10 mV (pH 6) and −179±10 mV (pH 8.5) vs NHE determined for the quinone/semiquinone potential of the C69A flavodoxin mutant by EPR titration [29].

In conclusion, this example gives a proof of principle for the fluorescence detection of a protein's redox state based on resonance energy transfer from an attached fluorescent label to the prosthetic group of the redox protein. This method permits not only to distinguish between the fully oxidized and fully reduced state of the protein but to estimate the degree of protein reduction or oxidation in the sample at submicromolar concentration. It can be potentially applied to any prosthetic group in a redox protein that changes its absorption spectrum upon reduction/oxidation, provided that a fluorescent label with a suitable fluorescence spectrum and a proper label attachment point can be chosen.

### EXAMPLE 5

Methylamine dehydrogenase (MADH) from Paracoccus denitrificians is a Tryptophan tryptophylquinone (TTQ) dependent dimeric enzyme that catalyses the reaction of methylamine to formaldehyde. The two electrons that are produced during the conversion of a methylamine molecule are transferred via 2 two consecutive one-electron steps from the TTQ cofactor of MADH to its physiological partner.

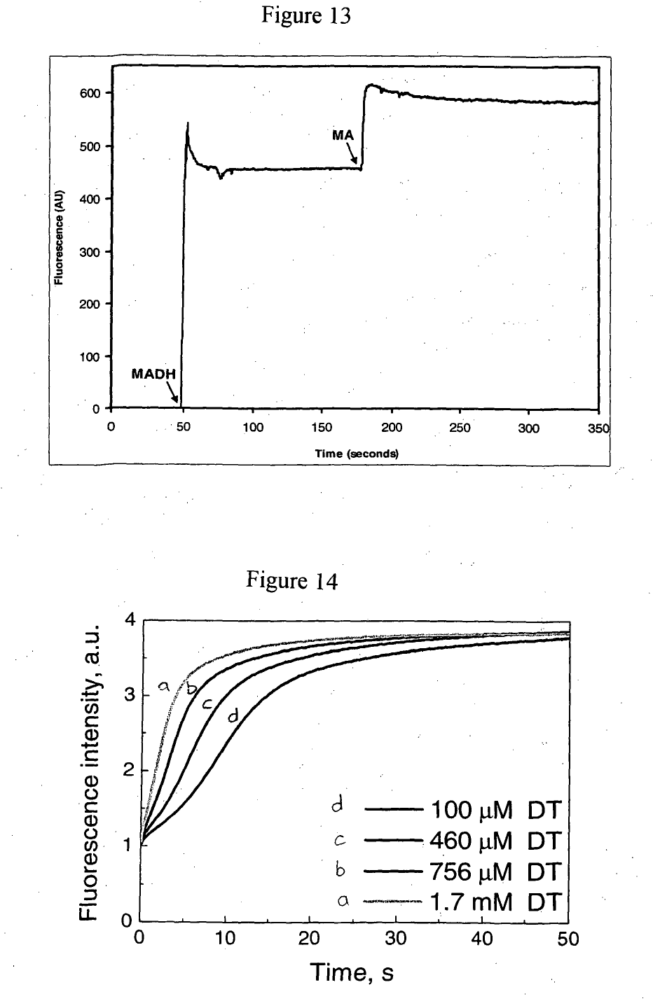

MADH was labeled on the N-terminus using Cy5 succinimidylester and the fluorescence intensity of the dye has been followed over time. The concentration of initially oxidized enzyme in this experiment was 4.4 μM in 20 mM Hepes buffer at pH 7.5. Clearly upon addition of 100 μM of methylamine (MA) the fluorescence increased (by approximately 25%), which can be attributed to loss in FRET efficiency from the dye to the prosthetic group of the enzyme in its reduced state (FIG. 13).

### EXAMPLE 6

Nitrite reductase (NiR) from Alcaligenes faecalis is a trimeric enzyme, of which each subunit contains a type 1 and a type 2 Cu centre. Upon reduction NiR receives one electron, which enters the enzyme via the type 1 site. This is followed by fast transfer to the type 2 site, where the enzyme converts nitrite into nitric oxide. NiR was labeled with Cy5 on position 93, which has been mutated into a cysteine group using Cy5 maleiimide. The labeling efficiency has been checked by absorption, which was approximately (data not shown) 55%.

Stopped flow experiments were performed under anaerobic conditions to look at the reduction kinetics of NiR following the fluorescence intensity of the label. The enzyme concentration was 2 μM in 20 mM Hepes buffer at pH 6.0 for these experiments whereas the concentration of reductant (sodiumdithionite) has been varied (from 1.7-100 μM) (FIG. 14).

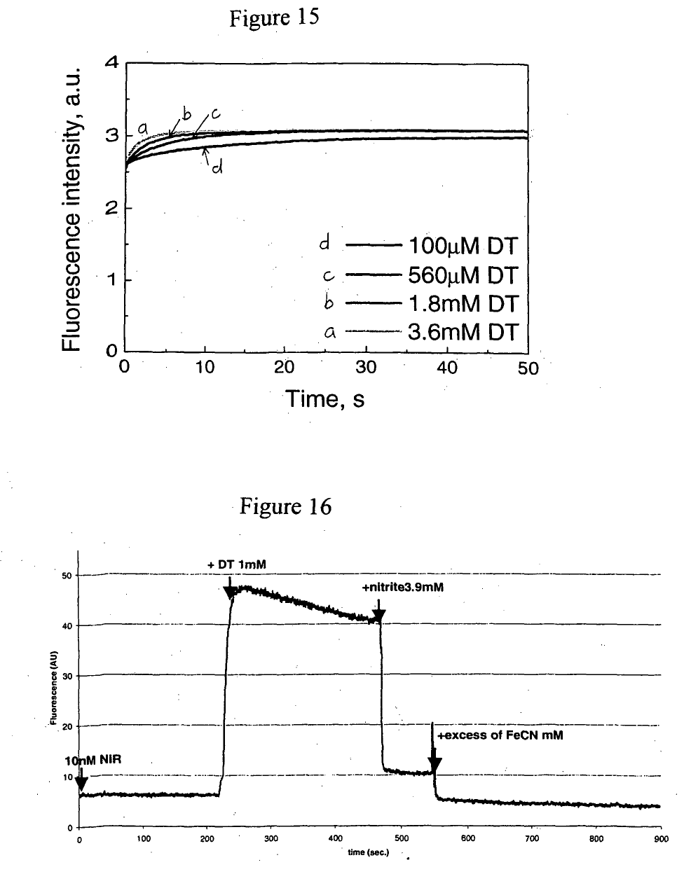

As a control the experiment was repeated with another mutant, where the type1 site is supposed to stay in the reduced form (FIG. 15).

These experiments show that, in the first case, the reduction event occurring at the type 1 site is being observed. It can not be excluded, however, that the type 2 site also influences the fluorescence intensity of the label, because there is still a (small) effect obtained in case of the redox inactive enzyme.

### EXAMPLE 7

In another experiment nitrite reductase was labeled again on position 93 using Cy5 maleiimide and the turnover of nitrite was monitored. A time course was performed, in which the fluorescence intensity was studied again as a function of time. The concentration of initially oxidized enzyme in this experiment was 10 nM in 50 mM Hepes/50 mM MES buffer at pH 6.0. First NiR was reduced using excess of sodiumdithionite (1 mM), which was followed by addition of 3.9 mM of nitrite. Finally the enzyme was fully oxidized by addition of 1 mM sodium ferricyanide (FeCN). This experiment was performed under anaerobic conditions (FIG. 16).

Clearly the reduced labeled enzyme could be converted into its oxidized state by addition of nitrite which initialized the start of enzyme turnover. This gave a huge quenching of the fluorescence (more than 90%). The slow decrease in time of the fluorescence after reduction is due to oxygen leakage.

### EXAMPLE 8

SLAC (Small Laccase) is a multicopper oxidase containing a type-1 Cu (T1) centre and a type-4 trinuclear Cu (T4) cluster. Laccase couples the four-electron reduction of oxygen with four consecutive one-electron oxidations of a substrate. The substrate specificity is low, many compounds that readily donate an electron (e.g. many phenols) are oxidized. This makes the laccase enzymes a versatile general oxidant.

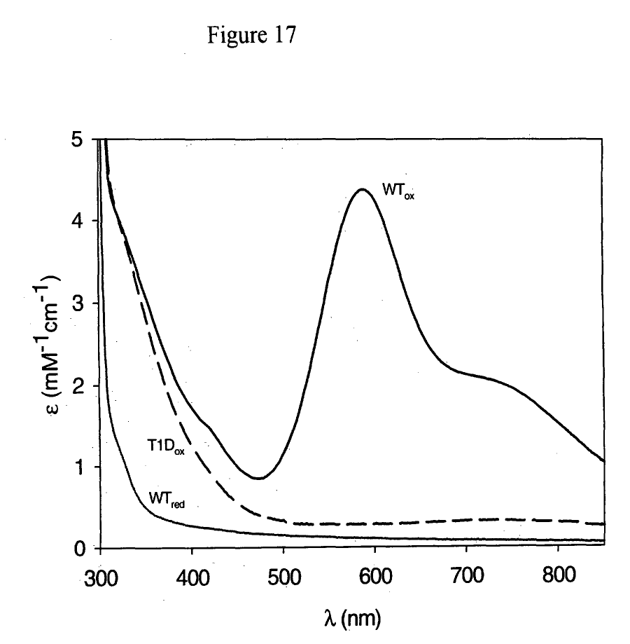

The oxygen chemistry takes place at the T4 cluster, while the T1 site is the entry point of the electrons donated by the substrate. The optical absorption spectrum is characterized by main bands at 330 and 590 nm and a weaker very broad feature around 750 nm (FIG. 17). All spectra were recorded in 100 mM Pᵢ buffer at pH 6.80 and at room temperature. The absorptions associated with the oxidised enzyme disappear when the protein is reduced. The 330 nm band originates from the T4 centre, while the 590 and 750 nm bands are associated with the T1 centre.

The endogenous tryptophan (Trp) fluorescence of SLAC (excitation 280-290 nm, emission 330-340 nm) is sensitive to the SLAC oxidation state. The Trp fluorescence increases by a factor of about two upon going from oxidized to fully reduced. The Trp fluorescence reflects the oxidation state of the trinuclear (T4) centre. This is in line with a possible energy transfer between excited Trp and the absorption at 330 nm of the T4 centre in the oxidized form. Thus, the tryptophan residues can be regarded as ‘natural labels’ that sense the oxidation state of the three Cu ions in the T4 cluster.

### EXAMPLE 9

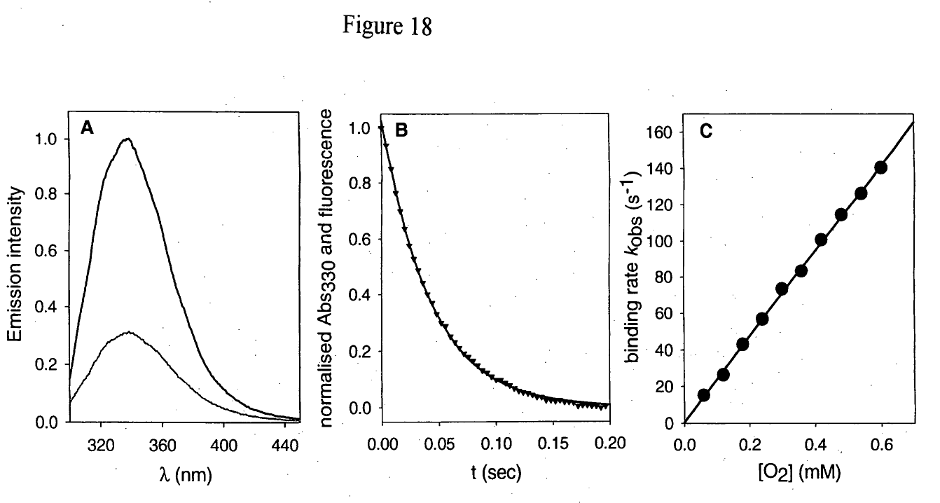

The principle of example 8 can be used to selectively determine the oxidation state of the T4 cluster as a function of enzyme activity. For example, it has been used to obtain insight into the reaction of reduced SLAC with molecular oxygen (FIGS. 18B and 18C). These figures show that SLAC tryptophan fluorescence reflects the oxidation state of the T4 Cu cluster. In B, the decrease in TrP emission intensity when reduced SLAC (1 μM) is mixed with O₂ (0.13 mM) is shown. A reaction rate can be extracted from these data. In this example, k=21 s⁻¹. The latter is of key importance in the understanding of the catalytic mechanism.

Next to the near-UV absorption of the T4 cluster, the optical absorption spectrum of the enzyme also shows the typical strong ‘blue’ absorption of the T1 centre, which shows a maximum at 590 nm (FIG. 17). This absorption can be used as a Förster acceptor for the emission of a synthetic label. The labeling of SLAC with a fluorescent label sensitive to the oxidation state of the T1 centre provides the perspective of being able to follow the T4 and T1 cluster on the same sample. This, in turn, provides a handle on the poorly understood catalytic mechanism of the laccases. It also opens the possibility to study the enzyme on a single molecule level. The feature could further be used to monitor the activity of ‘catalytic amounts’ of laccase (nM), which could be valuable in monitoring industrial bleaching reactions or in the development of biosensors for phenolic compounds (e.g. wastewater monitoring).

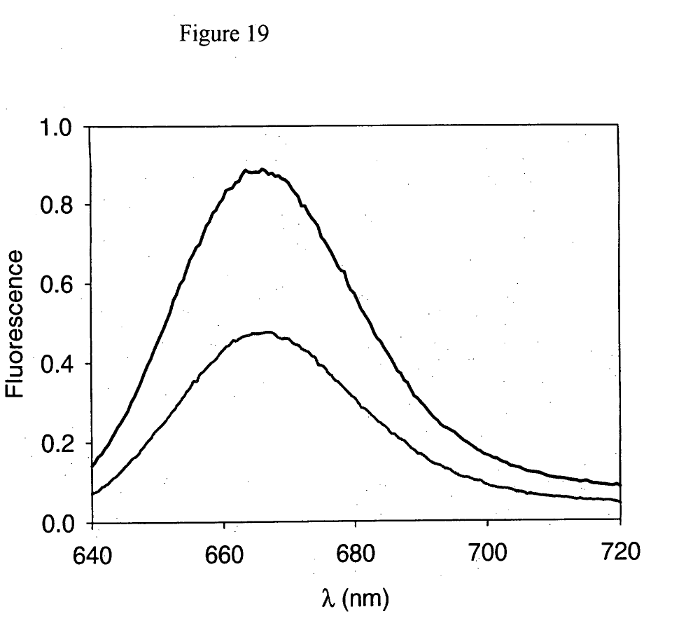

FIG. 19 shows the emission spectra of SLAC N-terminally labelled with the Cy5 flurophore. The fluorophore emits around 665 nm and is quenched by the absorption of the oxidised T1 Cu. The emission intensity differs by a factor about two between oxidised and reduced protein.

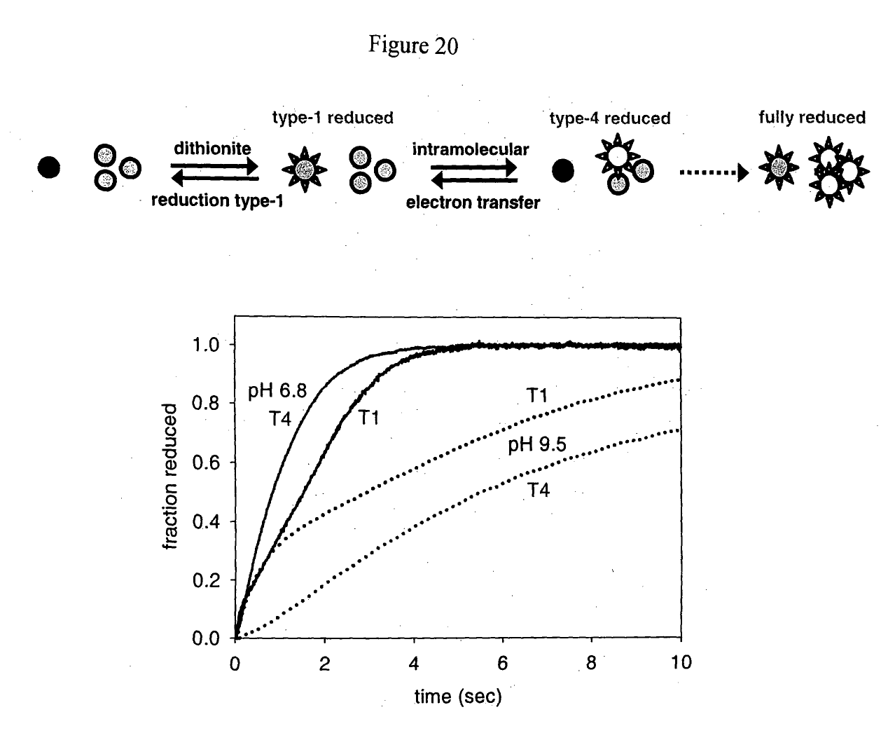

Thus, the endogenous Trp fluorescence combined with Cy5 labeling provides a system in which the oxidation state of the T1 site and the T4 cluster can be monitored independently. As a preliminary test case, the reduction of oxidised SLAC (1 μM) by dithionite (1 50μM) was studied at two pH values (FIG. 20) under anaerobic conditions using stopped-flow fluorescence spectroscopy. The endogenous Trp fluorescence reflects the oxidation state of the T1 site. In time, the SLAC is progressively reduced by dithionite, resulting in an increase in the Trp and label fluorescence intensity. It is immediately apparent that the Trp and the Cy5 fluorescence demonstrate different kinetics, showing that the ‘double labeling’ concept works. At low pH, the T4 site is reduced earlier than the T1 site, showing that the electron transfer from the T1 to the T4 cluster is fast. The reverse is observed at high pH, while the reduction is slower than at low pH. Both observations point towards a rate-limiting intra-molecular electron-transfer step at high pH.

### EXAMPLE 10

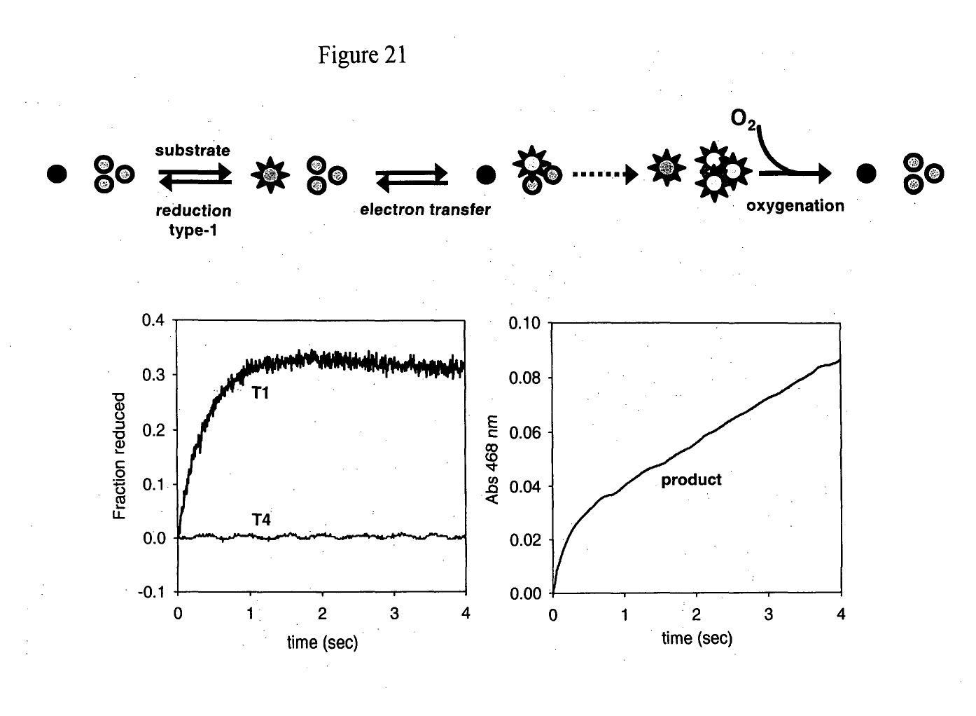

In another experiment, the SLAC oxidation state was monitored during the turnover of the substrate 2,6-dimethoxyphenol (DMP) at pH 9.5, again using a low concentration of (1 μM) SLAC, using stopped-flow flurosence spectroscopy. FIG. 21 shows the approach to the steady-state in SLAC catalysed turnover of 2,6-dimethoxyphenol. Around 1 μM oxidised Cy5 labelled SLAC was mixed with 0.5 mM DMP under aerobic conditions (0.2 mM O₂) at pH 9.5, after which Trp fluorescence (T4 cluster), Cy5 flurosence (T1 site) and the absorption at 468 nm (product) were monitored. The oxidation product of DMP is bright orange with an absorption maximum at 462 nm. This allows for the monitoring of product formation in addition to the T1/T4 oxidation states. It is the combination of these data that is crucial in obtaining a detailed understanding of the laccase mechanism.

The first two seconds of the reaction (FIG. 21) represent the approach to a steady-state. This steady-state reflects the equilibrium between different enzyme states during turnover and provides information on the rate-limiting step(s) in the catalytic conversion. It appears that the T4 cluster is fully oxidised in the steady-state, showing that the reaction with O₂ is not rate-limiting. Instead, a significant fraction of the T1 copper is reduced, again pointing towards a slow electron-transfer from the T1 Cu to the T4 cluster. The product formation shows so-called ‘burst kinetics’, indicating a rate-limiting step after substrate oxidation, which would be in line with the fluorescence data.

### REFERENCES

[1]=Cote, G. L. et al (2003) IEEE Sensors J. 3, 251 Emerging biomedical sensing technologies and their applications.
[2]=Stryer et al (1967) Proc Natl Acad Sci USA 58, 719-726 Energy transfer: a spectroscopic ruler.
[3]=Kenworthy, A. K. (2001) Methods 24, 289-296 Imaging protein-protein interactions using fluorescence resonance energy transfer microscopy.
[4]=Matayashi, E. D. et al (1990) Science 247, 954-958 Novel Fluorogenic substrates for assaying retroviral proteases by resonance energy transfer.
[5]=Tyagi, S et al (1996) Nat Biotechnol 14, 303-308 Molecular beacons: probes that fluorescence upon hybridisation.
[6]=Horn et al, EP 1445602
[7]=Tsien et al, WO 96/2381.
[8]=Lakawicz, J. R. (1999) Principles of Fluorescence Spectroscopy (2nd edition), Kluwer/Plenum, New York, Chap 13, page 367-391.
[9]=Wijma et al (2003), Biochemistry 42:4075.
[10]=Nar, H et al(1991) J. Mol Biol. 218, 427-447.
[11]=Van Amsterdam et al (2002) Inorg. Chim. Acta 331, 296-302.
[12]=Van de Kamp, M (1990) Biochemica et Biophysica Acta 1019, 283-292.
[13]=Jocelyn, P. C (1987) Method Enzymo 143, 246-256 Chemical reduction of disulfides.
[14]=Mujumbar, R. B. (1993) Bioconjugate Chem. 4,105-111.
[15]=Ubbink, M. et al (1992) Journal of Bacteriology 174, 3707-3714.
[16]=Klugkist, J. et al (1986) European Journal of Biochemistry 155, 33-40.
[17]=Impagliazzo, A et al (2004) Journal of Biomolecular Nmr 29, 541-542.
[18]=Kalverda, A. P. (1994) et al Solution Structure of the Type-1 Blue Copper Protein Amicyanin from Thiobacillus-Versutus, Journal of Molecular Biology 240, 358-371.
[19]=Kohzuma, T et al (1999) Journal of Biological Chemistry 274, 11817-11823.
[20]Buschmann, V et al (2003) Bioconjugate Chemistry 14,195-204.
[21]=Kolczak et al (2001) in Handbook of Metalloproteins (Messerschmidt, A., Huber, R., Poulos, T., and Wieghardt, K., Eds.), pp. 1170-1194, John Wiley & Sons, Chichester.
[22]=Lommen, A et al (1990) European Journal of Biochemistry 192, 653-661.
[23]=Dutton, P. L. (1978) Methods Enzymol. 54, 411-435.
[24]=St Clair, C. S. et al (1992) Inorganica Chimica Acta 191, 149-155.
[25]=Worrall, J et al (2005) FEBS Journal 272, 2441-2455.
[26]=Yoch, D. C. et al (1972) Annual Review of Microbiology 26, 139-162.
[27]=Hinkson, J. W. et al (1967) Journal of Biological Chemistry 242, 3345-3351.
[28]=Alagaratnam, S et al (2005) A Crystallographic Study of Cys69Ala Flavodoxin II of Azotobacter vinelandii: Structural Determinants of Redox Potential, Protein Science.
[29]=Steensma, E et al (1996) European Journal of Biochemistry 235, 167-172.

1. A method of fluorescence detection of a donor-acceptor pair in which a labelled protein comprising a fluorescent energy donor label and at least one energy acceptor moiety capable of accepting the energy from the donor label by Frster energy transfer is exposed to incident electromagnetic energy to excite the donor moiety and the fluorescence emission of the donor is measured, characterised in that the or each energy acceptor moiety has a more active and less active energy acceptor state and in that the level of quenching of fluorescence is indicative of the state of the energy acceptor moiety.

2. The method according to claim 1 in which the or each energy acceptor moiety may be reversibly converted from its more active state to its less active energy state.

3. The method according to claim 1 in which the or each energy acceptor moiety may be converted from its more active energy acceptor state to its less active energy acceptor state by a redox reaction.

4. The method according to claim 1 in which the or each energy acceptor moiety may be converted from its less active energy acceptor state to its more active energy acceptor state by a redox reaction.

5. The method according to claim 3 in which the redox reaction involves a redox partner protein accepting or donating electrons to the labelled protein via docking with the labelled protein.

6. The method according to claim 5 in which the redox partner protein is an enzyme capable of oxidising or reducing substrates and which method is carried out in the putative presence of such a redox substrate whereby the level of quenching of fluorescence is indicative of the extent of the enzymic redox reaction.

7. The method according to claim 6 in which the redox partner protein comprises its own fluorescent energy donor label and energy acceptor moiety, which dye fluoresces at a different wavelength to that on first protein and electron relay between partner proteins can be monitored by dual wavelength detection.

8. The method according to claim 3 in which the or each energy acceptor is a metal ion containing cofactor, which metal ion has two oxidation states, one of which is the less active energy acceptor state and the other of which is the more active energy acceptor state.

9. The method according to claim 8 in which the metal is copper or iron.

10. The method according to claim 8 in which the protein is azurin, a member of the blue copper protein family, or a conjugate thereof which is a fusion protein.

11. The method according to claim 8 in which the protein is a haem-containing protein.

12. The method according to claim 6 in which the protein is pseudo-azurin, a member of the azurin-like family, or a conjugate thereof which is a fusion protein and the partner protein is nitrite reductase.

13. The method according to claim 1 in which the protein is Nitrite reductase or Small Laccase.

14. The method according to claim 3 in which the or each energy acceptor is an organic co-factor.

15. The according to claim 14 in which the organic co-factor is flavin.

16. The method according to claim 14 in which the protein is methylamine dehydrogenase.

17. The method according to claim 3 in which electrodes are used as a source and/or sink of electrons for the redox process.

18. The method according to claim 17 wherein the protein is immobilised on the electrode surface.

19. The method according to claim 18 whereby the electrode is made of glass and total internal reflection is used to excite the surface-bound protein molecules.

20. The method according to claim 1 in which the label is selected from Cy5, Cy3, Alexa Fluor (488, 568, 594 or 647), TMR and Texas Red.

21. The method according to claim 1 in which there is substantially no change in the distance or relative orientation of the donor label and acceptor moiety when the acceptor is converted between the less active and more active energy absorbing states.

22. The method according to claim 1 in which the label is conjugated to a cysteine residue of the protein, optionally through a linker.

23. A labelled protein comprising a fluorescent energy donor label and at least one energy acceptor moiety capable of accepting energy from the donor label by Frster energy transfer characterised in that the or each energy acceptor moiety has a more active and a less active energy acceptor state between which the moiety may be converted.

24. The protein according to claim 23 in which the or each energy acceptor moiety may be reversibly converted from its more active state to its less active energy state.

25. The protein according to claim 23 in which the distance between and relative orientations of the label and the or each each energy acceptor moiety remain substantially unchanged during conversion of the acceptor moiety between the more active and less active energy accepting status.

26. The protein according to claim 23 in which the or each energy acceptor moiety is converted from its more active energy acceptor state to its less active energy acceptor state by a redox reaction.

27. The protein according to claim 23 in which the or each energy acceptor moiety is converted from its less active energy acceptor state to its more active energy acceptor state by a redox reaction.

28. The protein according to claim 23 in which the or each energy acceptor is a metal ion containing cofactor, which metal ion is convertible between two oxidation states, one of which is the less active energy acceptor state and the other of which is the more active energy acceptor state.

29. The protein according to claim 28 in which the metal is copper or iron.

30. The protein according to claim 28 in which the protein is azurin, a member of the blue copper protein family, or a conjugate thereof which is a fusion protein.

31. The protein according to claim 28 which is a haem-containing protein.

32. The protein according to claim 28 which is Nitrite reductase or Small laccase.

33. The protein according to claim 23 in which the or each energy acceptor is an organic cofactor.

34. The protein according to claim 33 in which the organic cofactor is flavin.

35. The protein according to claim 34 which is methylamine dehydrogenase.

36. The protein according to claim 23 in which the label is selected from Cy5, Cy3, Alexa Fluor (488, 568, 594 or 647), TMR and Texas Red.

37. A system comprising a protein according to claim 24 and a redox partner protein, a light source for imposing incident light at the excitation wavelength for the fluorescent label and a light detector capable of detecting the fluorescence emitted by the label.

38. A system comprising a protein according to claim 24 in contact with electrodes.

39. The system according to claim 38 in which the protein is immobilised on the electrode surface.

40. The system according to claim 38 in which the electrode is transparent to light of wavelength for exciting the fluorescent label and to the fluorescence emitted by the label.
